(19) United States
(12) Patent Application Publication
(10) Pub. No.: US 2025/0017786 A1
(43) Pub. Date: Jan. 16, 2025

(54) Elastomeric Bandage Device

(71) Applicant: Jon Keller, Firestone, CO (US)

(72) Inventor: Jon Keller, Firestone, CO (US)

(21) Appl. No.: 18/222,183

(22) Filed: Jul. 14, 2023

(51) Int. Cl. A61F 13/02

(52) U.S. Cl. CPC A61F 13/0246; A61F 13/0206; A61F 2013/00131

(57) ABSTRACT

An elastomeric bandage device includes an elastomeric bandage that can be wrapped around a user's body such that the elastomeric bandage extends over a wound in the user's body. An adhesive layer is applied to the elastomeric bandage to adhere to the user's body when the elastomeric bandage is wrapped around the user's body. A pad is attached to the elastomeric bandage at a connection point such that the pad is inhibited from being stretched when the elastomeric bandage is stretched. In this way the pad remains positioned over the wound when the elastomeric bandage is wrapped over the wound. The pad is comprised of a fluid absorbent material to absorb bodily fluids from the wound.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to bandage devices and more particularly pertains to a new bandage device for covering a wound without restricting movement of the user. The device includes an elastomeric bandage and a pad attached to the elastomeric bandage at a single connection point to facilitate the pad to remain motionless when the elastomeric bandage is stretched. In this way the elastomeric bandage can be securely wrapped around the user's hand, for example, while facilitating the pad to remain aligned with a wound on the user's hand.

### (2) Description of Related Art Including Information Disclosed Under 37 CFR 1.97 and 1.98.

The prior art relates to bandage devices including a variety of bandage devices that each includes a bandage that is strategically structured to conform to a specific part of a user's body and an absorbent pad that is fixed to the bandage. In no instance does the prior art disclose an elastomeric bandage and a pad that is attached to the elastomeric bandage at a single connection point thereby facilitating the pad to remain motionless when the elastomeric bandage is stretched.

### BRIEF SUMMARY OF THE INVENTION

An embodiment of the disclosure meets the needs presented above by generally comprising an elastomeric bandage that can be wrapped around a user's body such that the elastomeric bandage extends over a wound in the user's body. An adhesive layer is applied to the elastomeric bandage to adhere to the user's body when the elastomeric bandage is wrapped around the user's body. A pad is attached to the elastomeric bandage at a connection point such that the pad is inhibited from being stretched when the elastomeric bandage is stretched. In this way the pad remains positioned over the wound when the elastomeric bandage is wrapped over the wound. The pad is comprised of a fluid absorbent material to absorb bodily fluids from the wound.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a bottom perspective view of an elastomeric bandage device according to an embodiment of the disclosure.

FIG. 2 is a bottom view of an embodiment of the disclosure.

FIG. 3 is a front view of an embodiment of the disclosure.

FIG. 4 is a detail view taken from circle 4 of FIG. 3 of an embodiment of the disclosure.

FIG. 5 is a perspective in-use view of an embodiment of the disclosure showing an elastomeric bandage being applied to a wound.

FIG. 6 is a perspective in-use view of an embodiment of the disclosure showing an elastomeric bandage being wrapped around a user's hand.

FIG. 7 is a perspective in-use view of an embodiment of the disclosure showing an elastomeric bandage being secured around a user's hand.

FIG. 8 is a perspective view of an alternative embodiment of the disclosure.

FIG. 9 is a front view of an alternative embodiment of the disclosure.

FIG. 10 is a cross sectional view taken along line 10-10 of FIG. 9 of an embodiment of the disclosure.

### DETAILED DESCRIPTION OF THE INVENTION

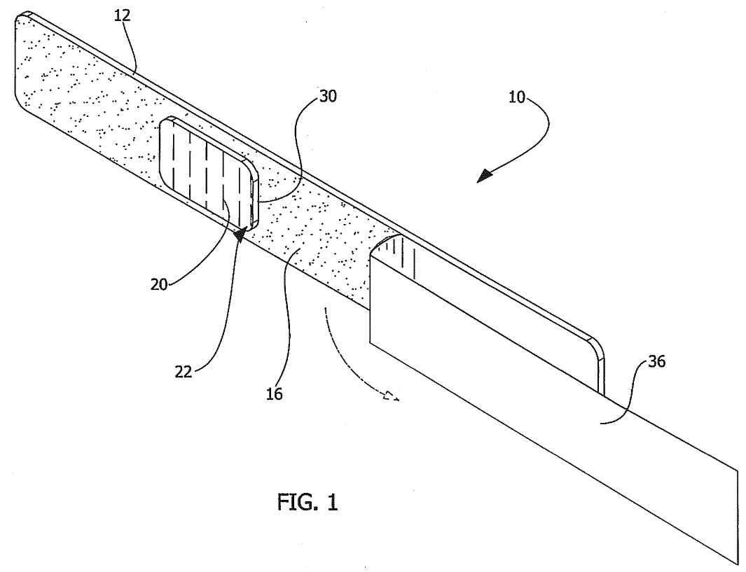

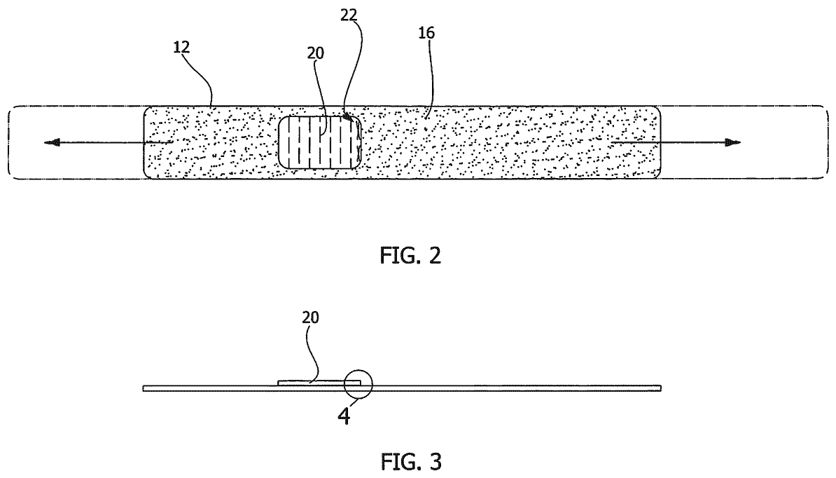

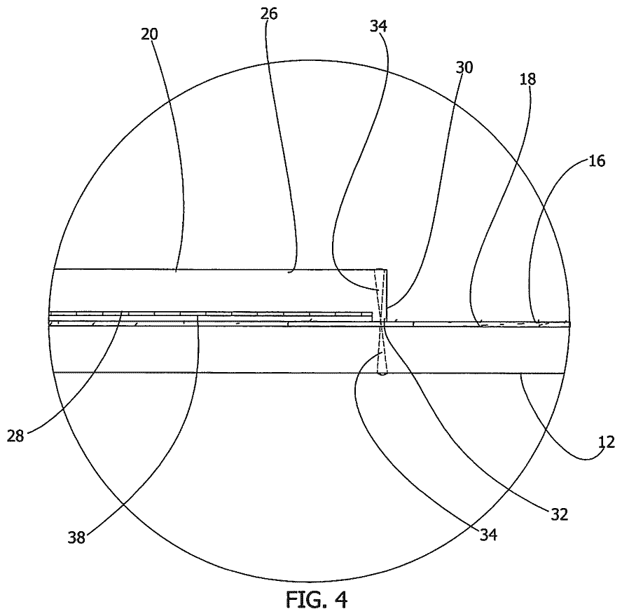

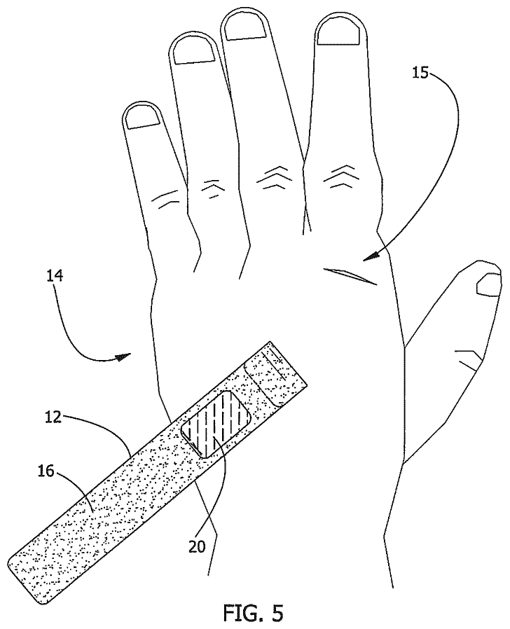

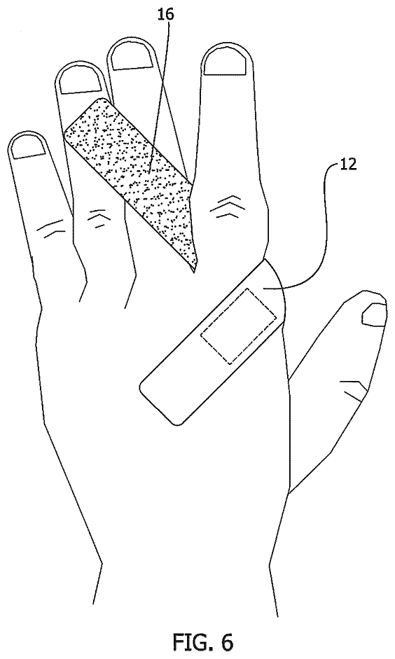

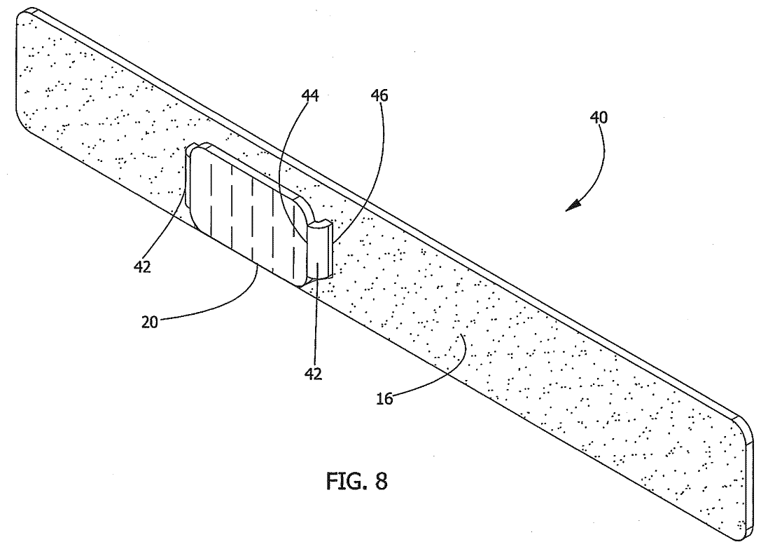

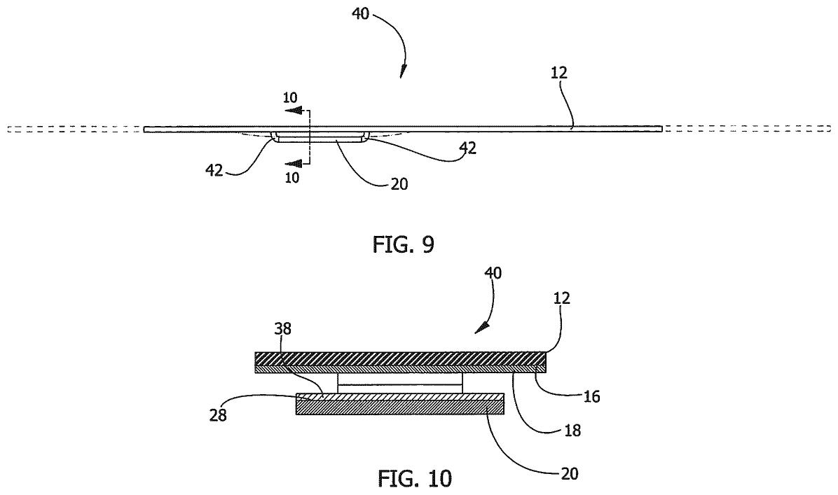

With reference now to the drawings, and in particular to FIGS. 1 through 10 thereof, a new bandage device embodying the principles and concepts of an embodiment of the disclosure and generally designated by the reference numeral 10 will be described.

As best illustrated in FIGS. 1 through 10, the elastomeric bandage device 10 generally comprises an elastomeric bandage 12 that can be wrapped around a user's body 14 such that the elastomeric bandage 12 extends over a wound 15 in the user's body 14. The elastomeric bandage 12 is stretchable such that the elastomeric bandage 12 can be secured on the user's body 14. An adhesive layer 16 is applied to the elastomeric bandage 12 to facilitate the adhesive layer 16 to adhere to the user's body 14 when the elastomeric bandage 12 is wrapped around the user's body 14. The adhesive layer 16 completely covers a bottom surface 18 of the elastomeric bandage 12.

A pad 20 is attached to the elastomeric bandage 12 at a connection point 22 such that the pad 20 is inhibited from being stretched when the elastomeric bandage 12 is stretched. In this way the pad 20 remains positioned over the wound 15 when the elastomeric bandage 12 is wrapped and stretched over the wound 15. The pad 20 is comprised of a fluid absorbent material, including but not being limited to cotton or other textile, to absorb bodily fluids from the wound 15. The pad 20 has a primary surface 26, a secondary surface 28 and a coupled end 30 extending between the primary surface 26 and the secondary surface 28. The pad 20 has a foot 32 extending away from the secondary surface 28 and the foot 32 is oriented to extend along the coupled end 30. The foot 32 abuts the adhesive layer 16 on the elastomeric bandage 12 such that the secondary surface 28 is spaced from the adhesive layer 16. Additionally, the pad 20 has stitching 34 extending through the primary surface 26 and the foot 32 and into the elastomeric bandage 12 thereby defining the connection point 22. A protective sheet 36 is removably applied to the adhesive layer 16 such that the protective sheet 36 inhibits the adhesive layer 16 from adhering to a surface. The protective sheet 36 completely covers the adhesive layer 16 and the pad 20.

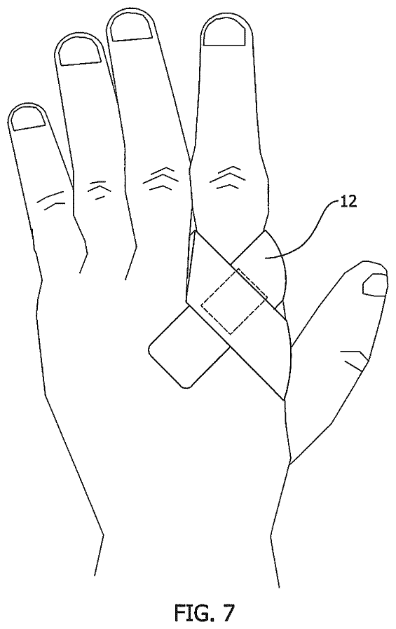

A protective layer 38 is provided and the protective layer 38 is applied to the secondary surface 28 of the pad 20. Additionally, the protective layer 38 completely covers the secondary surface 28. The protective layer 38 is comprised of a cohesive material such that the protective layer 38 resists adhering to the adhesive layer 16. In this way the pad 20 remains as free as possible from the elastomeric bandage 12 while still being secured to the elastomeric bandage 12. In an alternative embodiment 40 shown in FIGS. 7 through 9, a pair of couplers 42 is provided which each has a first end 44 and a second end 46. The first end 44 of each of the couplers 42 is attached to opposing ends of the pad 20. The second end 46 of each of the couplers 42 is coupled to the adhesive layer 16 such that each of the couplers 42 curves between the pad 20 and the adhesive layer 16. In this way the protective layer 38 is spaced from the adhesive layer 16. Additionally, each of the couplers 42 is comprised of a resiliently stretchable material to facilitate the pad 20 to remain motionless when the elastomeric bandage 12 is stretched.

In use, the protective sheet 36 is removed from the adhesive layer 16 and the elastomeric band is stretched and wrapped around the user's hand, for example, or other part of the user's body 14 that has been wounded such that the pad 20 is aligned with the wound 15. Furthermore, the pad 20 does not move when the elastomeric bandage 12 is stretched to facilitate the pad 20 to be positioned on the wound 15 and the pad 20 does not stretch along with the elastomeric bandage 12. The adhesive layer 16 adheres to the user as well as to the portion of the elastomeric bandage 12 that is wrapped over itself. In this way the elastomeric bandage 12 is retained on the user in a manner that does not restrict motion of the user.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An elastomeric bandage device for covering a wound without restricting movement of a user, said device comprising:
an elastomeric bandage being configured to be wrapped around a user's body such that said elastomeric bandage extends over a wound in the user's body, said elastomeric bandage being stretchable wherein said elastomeric bandage is configured to be secured on the user's body;
an adhesive layer being applied to said elastomeric bandage wherein said adhesive layer is configured to adhere to the user's body when said elastomeric bandage is wrapped around the user's body;
a protective sheet being removably applied to said adhesive layer such that said protective sheet inhibits said adhesive layer from adhering to a surface; and
a pad being attached to said elastomeric bandage at a connection point such that said pad is inhibited from being stretched when said elastomeric bandage is stretched wherein said pad is configured to remain positioned over the wound when said elastomeric bandage is wrapped over the wound, said pad being comprised of a fluid absorbent material wherein said pad is configured to absorb bodily fluids from the wound.

2. The assembly according to claim 1, wherein said pad has a primary surface, a secondary surface and a coupled end extending between said primary surface and said secondary surface, said pad having a foot extending away from said secondary surface, said foot being oriented to extend along said coupled end, said foot abutting said adhesive layer on said elastomeric bandage such that said secondary surface is spaced from said adhesive layer.

3. The assembly according to claim 2, wherein said pad has stitching extending through said primary surface and said foot and into said elastomeric bandage thereby defining said connection point.

4. The assembly according to claim 2, further comprising a protective layer being applied to said secondary surface of said pad, said protective layer completely covering said secondary surface, said protective layer being comprised of a cohesive material such that said protective layer resists adhering to said adhesive layer.

5. An elastomeric bandage device for covering a wound without restricting movement of a user, said device comprising:
an elastomeric bandage being configured to be wrapped around a user's body such that said elastomeric bandage extends over a wound in the user's body, said elastomeric bandage being stretchable wherein said elastomeric bandage is configured to be secured on the user's body;
an adhesive layer being applied to said elastomeric bandage wherein said adhesive layer is configured to adhere to the user's body when said elastomeric bandage is wrapped around the user's body, said adhesive layer completely covering a bottom surface of said elastomeric bandage;
a protective sheet being removably applied to said adhesive layer such that said protective sheet inhibits said adhesive layer from adhering to a surface;
a pad being attached to said elastomeric bandage at a connection point such that said pad is inhibited from being stretched when said elastomeric bandage is stretched wherein said pad is configured to remain positioned over the wound when said elastomeric bandage is wrapped over the wound, said pad being comprised of a fluid absorbent material wherein said pad is configured to absorb bodily fluids from the wound, said pad having a primary surface, a secondary surface and a coupled end extending between said primary surface and said secondary surface, said pad having a foot extending away from said secondary surface, said foot being oriented to extend along said coupled end, said foot abutting said adhesive layer on said elastomeric bandage such that said secondary surface is spaced from said adhesive layer, said pad having stitching extending through said primary surface and said foot and into said elastomeric bandage thereby defining said connection point; and
a protective layer being applied to said secondary surface of said pad, said protective layer completely covering said secondary surface, said protective layer being comprised of a cohesive material such that said protective layer resists adhering to said adhesive layer.

6. The assembly according to claim 5, further comprising a pair of couplers each having a first end and a second end, said first end of each of said couplers being attached to opposing ends of said pad, said second end of each of said couplers being coupled to said adhesive layer such that each of said couplers curves between said pad and said adhesive layer, each of said couplers being comprised of a resiliently stretchable material to facilitate said pad to remain motionless when said elastomeric bandage is stretched.
